(19) United States
(12) Patent Application Publication
(10) Pub. No.: US 2015/0005649 A1
(43) Pub. Date: Jan. 1, 2015

(54) ELECTRONIC BLOOD PRESSURE METER AND CONTROL METHOD THEREOF

(71) Applicant: OMRON HEALTHCARE CO., LTD., Kyoto (JP)

(72) Inventors: Yoshihisa Miwa, Kyoto (JP); Takeyasu Hashimoto, Kyoto (JP)

(73) Assignee: OMRON HEALTH Co., Ltd., Kyoto (JP)

(21) Appl. No.: 14/372,404

(22) PCT Filed: Oct. 29, 2012
(86) PCT No.: PCT/JP2012/077856

§ 371 Date: Jul. 15, 2014

(30) Foreign Application Priority Data
Jan. 27, 2012 (JP) 2012-014746

(51) Int. Cl. A61B 5/0225; A61B 5/0235; A61B 5/021; A61B 5/022

(52) U.S. Cl. CPC A61B 5/0225; A61B 5/02233; A61B 5/0235; A61B 5/02225; A61B 5/02108
USPC 600/494

(57) ABSTRACT

A blood pressure meter includes an air bladder for attachment to a measurement area, a pressure sensor, a pump a valve, drive circuits, a CPU connected to the pressure sensor and the drive circuits, for controlling the adjustment of the internal pressure of the air bladder and calculating a blood pressure value based on a change in the internal pressure resulting from the adjustment, and a holding portion, into and from which air can flow from and to the air bladder, that has a lower volume than the air bladder. The air can flow from the holding portion into the air bladder in the case where a predetermined pressure is applied to the holding portion. The CPU starts the adjustment of the internal pressure in the case where a change in the internal pressure prior to the start of the adjustment of the internal pressure is a predefined change.

### TECHNICAL FIELD

This invention relates to electronic blood pressure meters and control methods thereof, and particularly relates to electronic blood pressure meters that measure blood pressures using a cuff and to control methods thereof.

### BACKGROUND ART

Managing a patient's blood pressure is one way in which a medical facility manages the patient on a regular basis. The patient's circulatory state can be understood by measuring the patient's blood pressure, which is a fundamental piece of each patient's biological information, over short intervals (normally every 2.5 to 5 minutes) during an operation, or measuring a patient's blood pressure periodically (two to three times a day, for example) while the patient is an inpatient in a hospital ward.

In operating rooms, hospital wards, and so on, blood pressure meters are often placed several meters away from the patient, such as at the head or foot of the patient's bed. As such, it is necessary for a medical worker or the like who is taking the measurement to first wrap a cuff around a measurement area such as the upper arm of the patient, and then move to the blood pressure meter to carry out operations for starting the measurement. This reduces the efficiency of the task.

### CITATION LIST

### Patent Literature

Patent Literature 1 International Publication Pamphlet No. 2005/074793

### SUMMARY OF INVENTION

### Technical Problem

The technique disclosed in Patent Literature 1 (International Publication Pamphlet No. 2005/074793), in which a blood pressure meter having an infrared light-based remote control function is used to carry out operations remotely, can be given as an example of an attempt to solve this problem.

However, such a device poses its own problem in that the device itself is expensive.

There is a further problem in that such a device increases a user's workload because the user must manage a remote controller, retrieve and operate the remote controller after wrapping the cuff on the upper arm or the like, and so on.

Having been achieved in light of such problems, it is an object of the present invention to provide an electronic blood pressure meter and a control method for an electronic blood pressure meter that enable efficient blood pressure measurement operations while also suppressing device costs.

### Solution to Problem

To achieve the aforementioned object, an electronic blood pressure meter according to an aspect of the present invention includes a fluid bladder that is attached to a measurement area of a measurement subject, a sensor for measuring an internal pressure in the fluid bladder, an adjustment mechanism for adjusting the internal pressure in the fluid bladder, a processing unit, connected to the sensor and the adjustment mechanism, for performing a process that controls the adjustment performed by the adjustment mechanism and calculates a blood pressure value based on a change in the internal pressure in the fluid bladder resulting from the adjustment, and a fluid holding portion, into and from which fluid can flow from and to the fluid bladder, that has a lower volume than the fluid bladder. The fluid can flow from the fluid holding portion into the fluid bladder in the case where a predetermined pressure is applied to the fluid holding portion. The processing unit starts the adjustment performed by the adjustment mechanism in the case where a change in the internal pressure of the fluid bladder detected by the sensor prior to the start of the adjustment by the adjustment mechanism is a predefined change.

Preferably, the processing unit calculates a predefined parameter from the change in the internal pressure of the fluid bladder detected by the sensor, and determines whether or not the change in the internal pressure of the fluid bladder detected by the sensor is the predefined change by comparing the calculated parameter with a parameter stored in advance based on the predefined change.

More preferably, the parameter is at least one of a number of pressure changes, a time interval of a pressure change, a degree of each pressure change, and a maximum pressure value or minimum pressure value in each pressure change.

Preferably, the processing unit determines that the change in the internal pressure in the fluid bladder detected by the sensor is the predefined change and starts the adjustment performed by the adjustment mechanism in the case where a number of changes in the internal pressure in the fluid bladder detected by the sensor is a number stored in advance, a time interval of the number of changes is greater than or equal to a specified time stored in advance, a degree of each change in the internal pressure in the fluid bladder detected by the sensor is greater than or equal to a degree of change stored in advance, and a maximum pressure value in each change in the internal pressure of the fluid bladder detected by the sensor is greater than or equal to a pressure value stored in advance.

A control method for a electronic blood pressure meter according to another aspect of the present invention is a control method for measuring a blood pressure using the electronic blood pressure meter. The electronic blood pressure meter includes a fluid bladder that is attached to a measurement area of a measurement subject and a fluid holding portion, into and from which fluid can flow from and to the fluid bladder, that has a lower volume than the fluid bladder, and the fluid is capable of flowing from the fluid holding portion into the fluid bladder in the case where a predetermined pressure is applied to the fluid holding portion. The control method includes a step of detecting a change in an internal pressure in the fluid bladder prior to the start of a blood pressure measurement operation, a step of comparing the change in the internal pressure in the fluid bladder with a predefined change, a step of starting control of the internal pressure in the fluid bladder in the case where the change in the internal pressure in the fluid bladder is the predefined change, a step of calculating a blood pressure value of the measurement subject based on the change in the internal pressure in the fluid bladder detected during the control of the internal pressure in the fluid bladder, and a step of outputting the calculated blood pressure value.

### Advantageous Effects of Invention

According to the invention, a blood pressure measurement operation can be carried out efficiently while also suppressing the cost of a device.

### BRIEF DESCRIPTION OF DRAWINGS

FIG. 1 is a block diagram illustrating a specific example of the configuration of an electronic blood pressure meter (simply “blood pressure meter” hereinafter) according to an embodiment.

FIGS. 2A and 2B are diagrams illustrating examples of the location of a holding portion.

FIG. 3 is a diagram illustrating a specific example of the configuration of the holding portion.

FIG. 4 is a block diagram illustrating a specific example of the functional configuration of the blood pressure meter.

FIGS. 5A and 5B are diagrams illustrating specific examples of changes in an internal pressure within an air bladder resulting from body movement in a measurement subject.

FIG. 6 is a diagram illustrating a specific example of a pattern of internal pressure changes for starting a measurement operation.

FIG. 7 is a flowchart illustrating a flow of operations performed in the blood pressure meter up to the start of a measurement operation.

FIG. 8 is a flowchart illustrating a flow of operations performed in the blood pressure meter up to the start of a measurement operation.

### DESCRIPTION OF EMBODIMENTS

Hereinafter, an embodiment of the present invention will be described with reference to the drawings. In the following descriptions, identical reference numerals are assigned to identical components and constituent elements. The names and functions thereof are also the same.

Apparatus Configuration

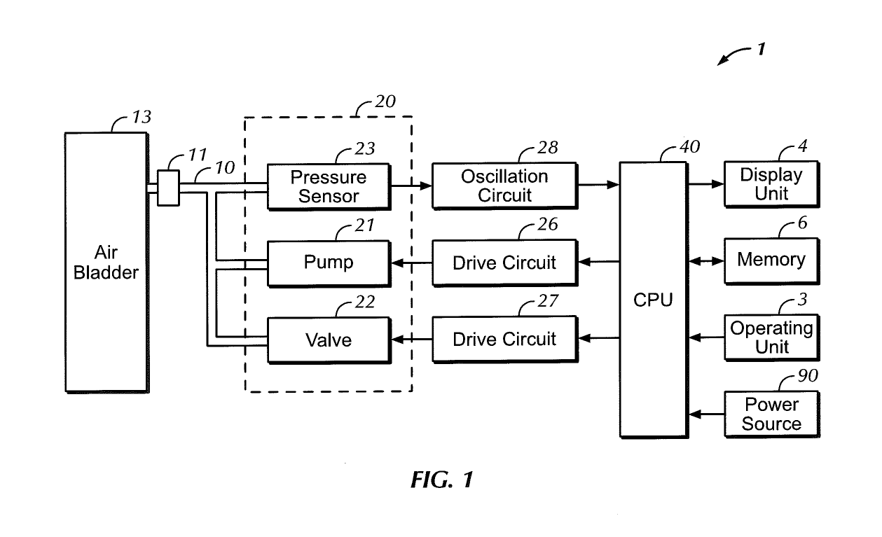

FIG. 1 is a block diagram illustrating a specific example of the configuration of an electronic blood pressure meter (simply “blood pressure meter” hereinafter) 1 according to the present embodiment.

As shown in FIG. 1, the blood pressure meter 1 includes an air bladder 13 for measurement, and the air bladder 13 is connected to an air system 20 via an air tube 10. The air system 20 includes a pressure sensor 23 for measuring an internal pressure of the air bladder 13, a pump 21 for supplying/exhausting air to/from the air bladder 13, and a valve 22.

The pressure sensor 23, the pump 21, and the valve 22 are respectively electrically connected to an oscillation circuit 28, a drive circuit 26, and a drive circuit 27; meanwhile, the oscillation circuit 28, the drive circuit 26, and the drive circuit 27 are all electrically connected to a CPU (central processing unit) 40 for controlling the blood pressure meter 1 as a whole.

Furthermore, a display unit 4, an operating unit 3, a memory 6 that holds information necessary for programs and calculations executed by the CPU 40, and a power supply 90 are connected to the CPU 40.

The drive circuit 26 drives the pump 21 in accordance with a control signal from the CPU 40. As a result, air is injected into the air bladder 13.

The drive circuit 27 drives the valve 22 in accordance with a control signal from the CPU 40. As a result, the valve 22 is opened/closed.

The pressure sensor 23 is an electrostatic capacitance-type pressure sensor, and an electrostatic capacitance value thereof changes as the internal pressure of the air bladder 13 changes. The pressure sensor 23 is connected to the oscillation circuit 28. The oscillation circuit 28 converts the electrostatic capacitance value of the pressure sensor 23 into a signal having an oscillation frequency and outputs the resulting signal to the CPU 40.

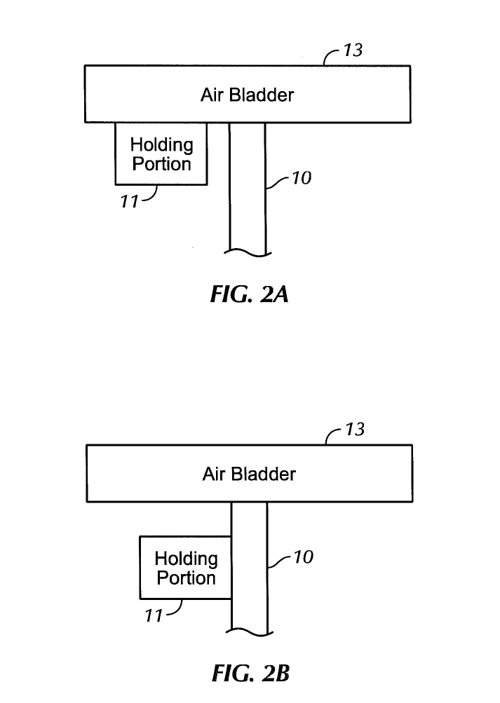

Furthermore, the blood pressure meter 1 includes a holding portion 11 that is associated with the air bladder 13 or the air tube 10. The holding portion 11 has an internal space for holding air, and holds a predetermined amount of air within that space. The holding portion 11 exhausts the air held therein when an external pressure of greater than or equal to a predetermined value is applied to the holding portion 11. The holding portion 11 may be disposed on the air tube 10, as shown in FIG. 2B, or may be disposed on the air bladder 13, as shown in FIG. 2A.

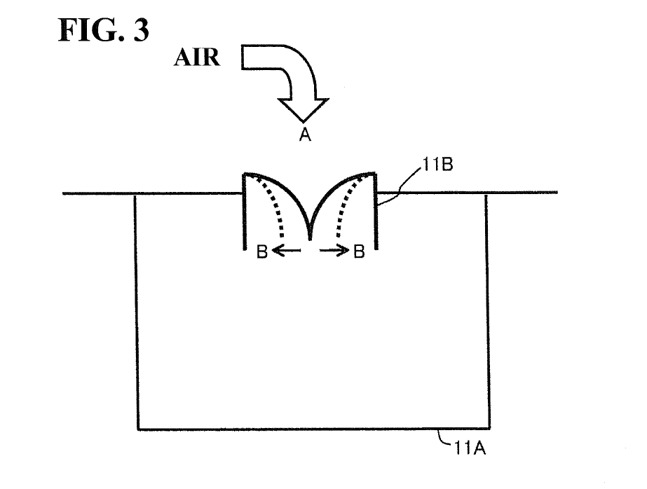

FIG. 3 is a diagram illustrating a specific example of the configuration of the holding portion 11.

As shown in FIG. 3, the holding portion 11 includes a holding space 11A for holding air within the holding portion 11, and a check valve 11B disposed at a border position between holding space 11A and the air tube 10 or the air bladder 13.

The size of the holding space 11A is not particularly limited as long as the volume thereof is sufficiently small compared to the air bladder 13. Specifically, the size of the holding space 11A may be small enough to avoid affecting the calculation of a blood pressure value based on an internal pressure change in the air bladder 13 while also being capable of holding an amount of air that produces an air pressure great enough to be detected by the pressure sensor 23.

According to this configuration, air within the air tube 10 or the air bladder 13 is introduced into the holding space 11A via the check valve 11B but is blocked by the check valve 11B and is thus not freely exhausted to the air tube 10 or the air bladder 13 from the holding space 11A, as indicated by an arrow A in FIG. 3. Accordingly, an amount of air equivalent to the volume of the space in the holding space 11A is held therein.

The check valve 11B opens slightly, as indicated by an arrow B in FIG. 3, when a given amount of pressure is applied from the holding space 11A side. Accordingly, the air in the holding portion 11 is exhausted to the air tube 10 or the air bladder 13 through the check valve 11B as a result of the given amount of pressure being applied to the holding portion 11.

The “given amount of pressure” assumes an amount of pressure applied by a person striking the holding portion 11.

Outline of Operations

Measurement operations of the blood pressure meter 1 start when an instruction to start the measurement is made after the air bladder 13 has been attached by wrapping a measurement band (not shown) containing the air bladder 13 around a measurement area such as a wrist, an upper arm, or the like.

The measurement operations involve controlling the internal pressure of the air bladder 13 so that the internal pressure of the air bladder 13 increases to predetermined pressure higher than a systolic blood pressure value of the measurement subject and then decreases. The CPU 40 calculates blood pressure values (the systolic blood pressure value, a diastolic blood pressure value, and the like) of the measurement subject based on pulse pressure variations superimposed on the changes in the internal pressure of the air bladder 13 during the pressure increase or decrease.

In the measurement performed by the blood pressure meter 1 according to the present embodiment, the aforementioned measurement operations are started by a measurer striking the measurement band after the measurement band has been wrapped around the measurement area. Accordingly, after wrapping the measurement band around the measurement area of the measurement subject, the measurer can instruct the measurement to start from the position where s/he wrapped the measurement band around the measurement area, without returning to the main body where the operating unit 3 is disposed.

Functional Configuration

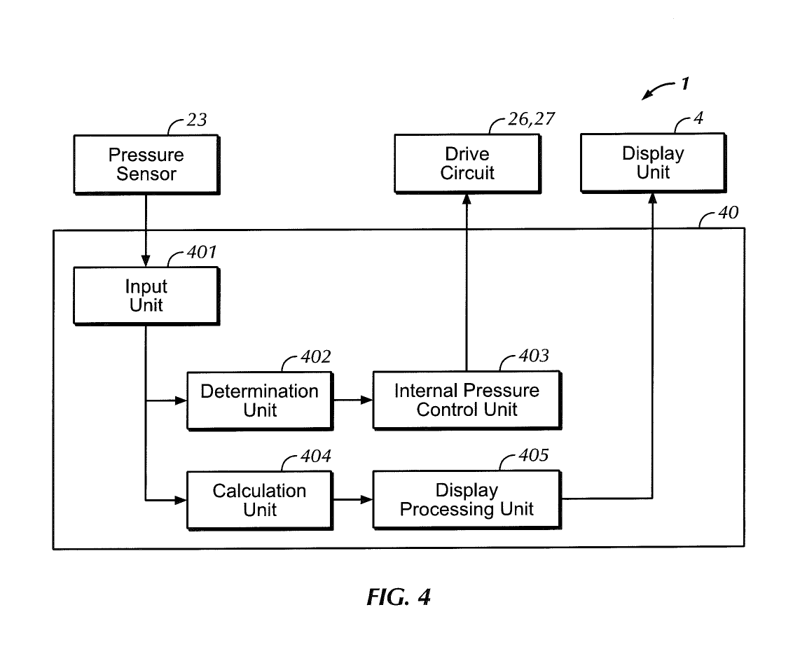

FIG. 4 is a block diagram illustrating a specific example of the functional configuration of the blood pressure meter 1 for carrying out the aforementioned operations. The respective functions indicated in FIG. 4 are realized primarily in the CPU 40, by the CPU 40 reading out programs from the memory 6 and executing those programs; however, at least some of the functions may instead be realized through hardware configurations such as the device configuration shown in FIG. 1, electric circuits, or the like.

As shown in FIG. 4, the CPU 40 includes an input unit 401 for accepting the input of a sensor signal from the pressure sensor 23; a determination unit 402 for determining whether or not to start measurement operations based on a change in the internal pressure of the air bladder 13, obtained from the sensor signal; an internal pressure control unit 403 for controlling the internal pressure of the air bladder 13 in accordance with the determination as to whether to start the measurement operations made by the determination unit 402; a calculation unit 404 for calculating a blood pressure value based on a change in the internal pressure of the air bladder 13 obtained from the sensor signal of the pressure sensor 23 while the internal pressure of the air bladder 13 is being controlled; and a display processing unit 405 for performing processing that displays the calculated blood pressure value in the display unit 4 as a measurement result.

Next, the determination made by the determination unit 402 will be described.

As described above, by applying pressure to the measurement band containing the air bladder 13 after the measurement band has been wrapped around the measurement area, the air held within the holding portion 11 is exhausted to the air tube 10 or the air bladder 13 via the check valve 11B, and the internal pressure in the air bladder 13 changes.

However, there are cases where pressure is continuously applied to the measurement band, such as when the measurement area is the upper arm and the arm around which the measurement band is wrapped ends up under the measurement subject's body due to the measurement subject turning over or the like. There are also cases where pressure is momentarily applied to the measurement band due to the measurement subject raising or lowering his/her arm or the like.

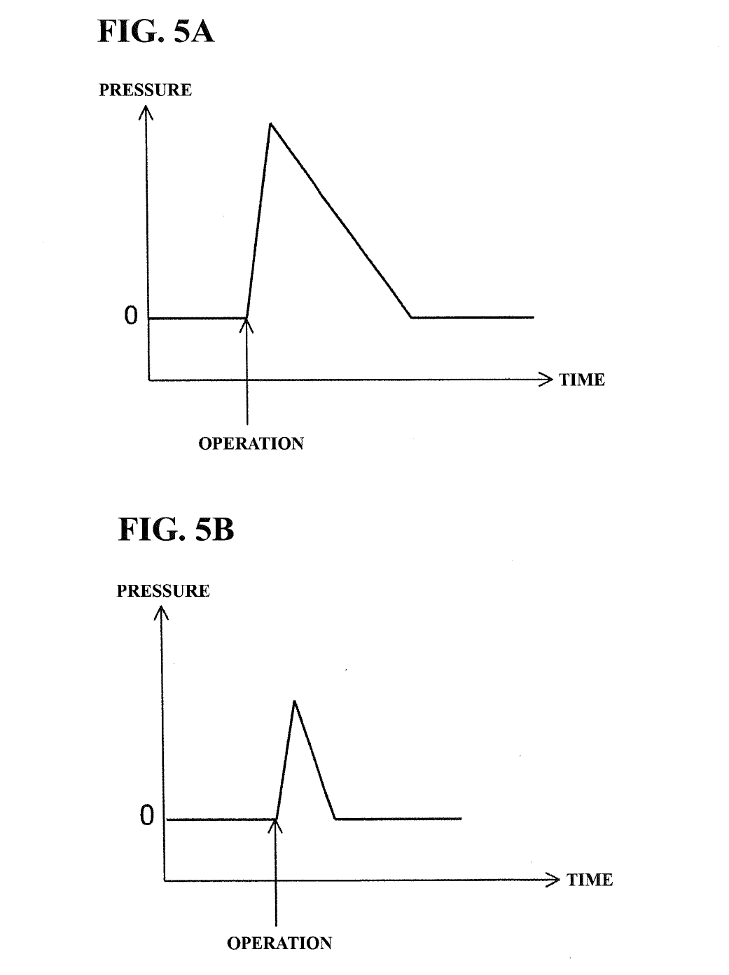

FIG. 5A illustrates an example of changes in the internal pressure of the air bladder 13 in the former case, whereas FIG. 5B illustrates an example of changes in the internal pressure of the air bladder 13 in the latter case.

There is thus a risk that erroneous operations will occur if measurement operations are simply started when there has been a change in the internal pressure of the air bladder 13.

Accordingly, the determination unit 402 stores a pattern of changes in the internal pressure in advance, and determines to start the measurement operations in the case where the measured pressure matches the pattern or has greater than or equal to a given amount of correlation with the pattern.

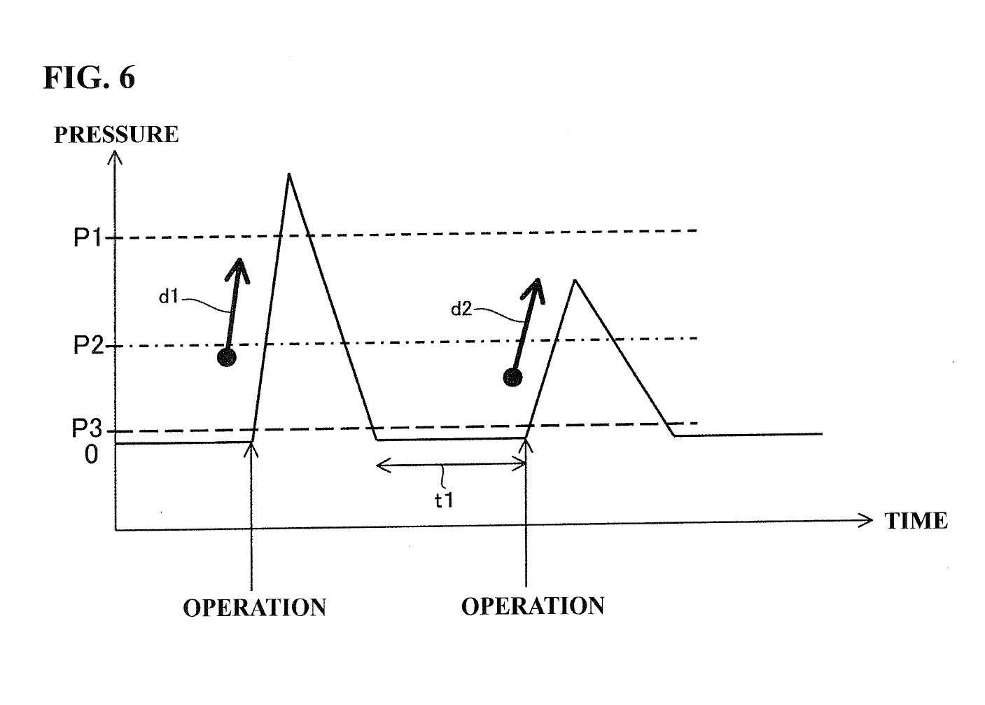

For example, the determination unit 402 may store an internal pressure change pattern such as that shown in FIG. 6. Parameters such as a number of times pressure is applied (a number of pressure changes), a time interval of changes, a degree of a change, a maximum pressure value/minimum pressure value, and so on can be given as example of the internal pressure change pattern. For example, all of these parameters may be stored, or at least one of these parameters may be stored.

In the example shown in FIG. 6, all of these parameters are stored; the number of times a pressure is applied is two times, and a time interval t1 thereof, the pressures applied each time (greater than or equal to a pressure P1, greater than or equal to a pressure P2 but less than P1), and a degree of change at each time (slopes d1 and d2) are defined as the parameters. The slopes d1 and d2 are indicated by arrows in FIG. 6.

The determination unit 402 calculates the respective parameters from the sensor signal obtained from the pressure sensor in the air bladder 13 over a predetermined period, and determines whether or not to start measurement operations by determining whether or not the parameters match corresponding parameters indicating a specified pressure change pattern or whether or not the parameters are within a predetermined range.

Note that in the following descriptions of a specific operational flow, it is assumed that a determination is made using the internal pressure change pattern shown in FIG. 6.

Flow of Operations

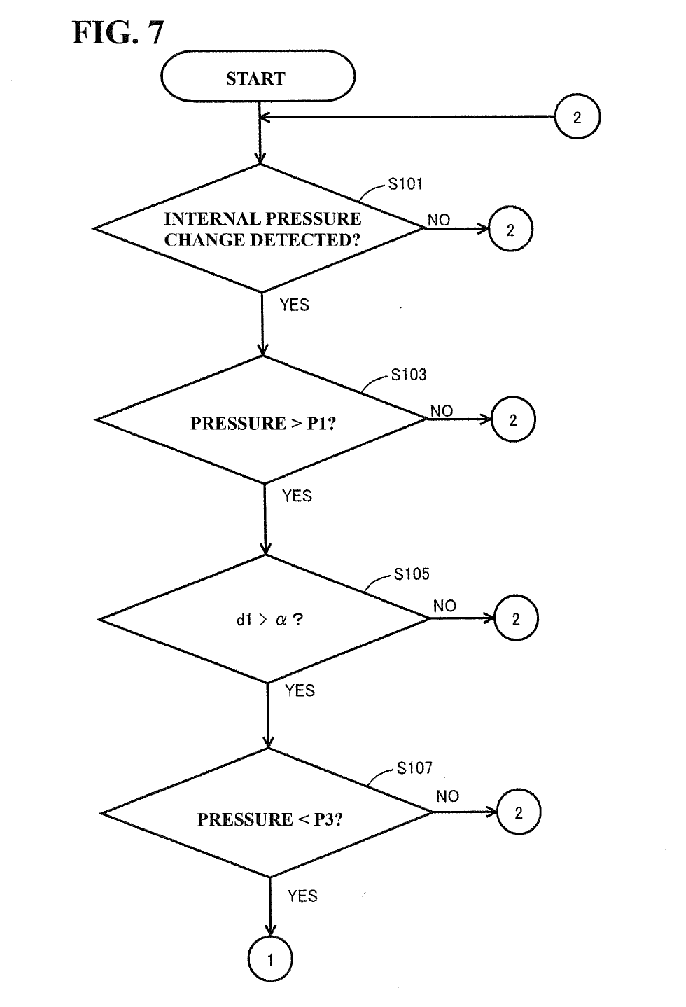

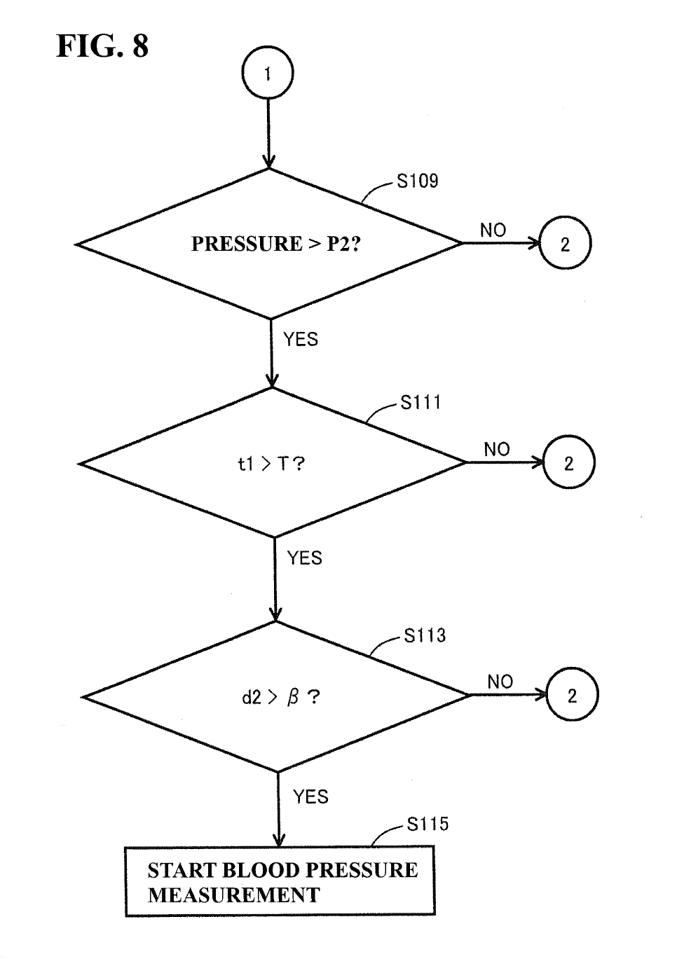

FIGS. 7 and 8 are flowcharts illustrating a flow of operations performed in the blood pressure meter 1 up to the start of a measurement operation. The operations indicated in the flowchart shown in FIGS. 7 and 8 are started when a power switch (not shown) included in the operating unit 3 is depressed and the blood pressure meter 1 is turned on, and are realized by the CPU 40 reading out and executing programs stored in the memory 6 and implementing the functions indicated in FIG. 4.

As shown in FIG. 7, when the CPU 40 detects a pressure change while standing by for measurement operations to start (YES in step S101), the CPU 40 calculates the respective parameters and compares the calculated parameters with the corresponding parameters for the specified pressure change. In other words, for the first pressure change, the CPU 40 calculates the pressure value thereof and determines whether or not that pressure value is greater than the specified pressure P1 (step S103); furthermore, the CPU 40 calculates the slope dl expressing the degree of that pressure change and determines whether or not the calculated slope dl is greater than a specified degree of change a (step S105). Then, the CPU 40 calculates the pressure value that follows thereafter, and determines whether or not the calculated pressure value has reached the pressure that is lower than a pressure P3 (step S107).

In the case where all of these conditions are met, the CPU 40 determines that the first specified pressure change pattern shown in FIG. 6 has occurred, and advances to the next determination (YES in steps S103 to S107).

As shown in FIG. 8, the CPU 40 calculates the respective parameters for the second pressure change, and compares the calculated parameters with the corresponding parameters for the specified pressure change pattern. In other words, the CPU 40 calculates the pressure value and determines whether or not the calculated pressure value is greater than the pressure P2 (step S109), and determines whether or not the time interval t1 from the previous pressure change is longer than a specified time T (step S111); furthermore, the CPU 40 determines whether or not the slope d2 expressing the degree of the pressure change is greater than a specified degree of change β (step S103).

In the case where all of these conditions are met, the CPU 40 determines that the second specified pressure change pattern shown in FIG. 6 has occurred (YES in steps S109 to S113). In other words, through this processing, the CPU 40 determines that the pressure change patterns shown in FIG. 6 have occurred and determines that the blood pressure measurement operations are to be started (step S115).

Note that in the case where the conditions of FIGS. 7 and 8 are not met (that is, the case where a determination of NO is made in steps S101, S103, S105, S107, S109, S111, and S113), the CPU 40 returns to a standby state, and repeats the processes of steps S101 and on.

### Effects of the Embodiment

By performing the aforementioned operations using the blood pressure meter 1, after wrapping the measurement band around the measurement area of the measurement subject, the measurer can instruct the measurement to start from the position where s/he wrapped the measurement band around the measurement area, without returning to the main body where the operation unit 3 is disposed. Furthermore, this effect can be achieved without requiring the blood pressure meter 1 to have any special configuration. Accordingly, the efficiency of the task can be greatly increased while also suppressing the cost of the device.

Note that the embodiment disclosed above is to be understood as being in all ways exemplary and in no way limiting. The scope of the present invention is defined not by the aforementioned descriptions but by the scope of the appended claims, and all changes that fall within the same essential spirit as the scope of the claims are intended to be included therein as well.

### REFERENCE SIGNS LIST

1 blood pressure meter

3 operating unit

4 display unit

6 memory

10 air tube

11 holding portion

11A holding space

11B check valve

13 air bladder

20 air system

21 pump

22 valve

23 pressure sensor

26, 27 drive circuit

28 oscillation circuit

40 CPU

90 power source

401 input unit

402 determination unit

403 internal pressure control unit

404 calculation unit

405 display processing unit

1. An electronic blood pressure meter comprising:
a fluid bladder for attachment to a measurement area of a measurement subject;
a sensor that measures an internal pressure in the fluid bladder;
an adjustment mechanism that adjusts the internal pressure in the fluid bladder;
a processing unit, connected to the sensor and the adjustment mechanism, that performs a process that controls the adjustment performed by the adjustment mechanism and calculates a blood pressure value based on a change in the internal pressure in the fluid bladder resulting from the adjustment; and
a fluid holding portion, into and from which fluid can flow from and to the fluid bladder, that has a lower volume than the fluid bladder,
wherein the fluid can flow from the fluid holding portion into the fluid bladder in a case where a predetermined pressure is applied to the fluid holding portion, and
wherein the processing unit starts the adjustment performed by the adjustment mechanism in a case where a change in the internal pressure of the fluid bladder detected by the sensor prior to the start of the adjustment by the adjustment mechanism is a predefined change.

2. The electronic blood pressure meter according to claim 1, wherein the processing unit calculates a predefined parameter from the change in the internal pressure of the fluid bladder detected by the sensor, and determines whether or not the change in the internal pressure of the fluid bladder detected by the sensor is the predefined change by comparing the calculated parameter with a parameter stored in advance based on the predefined change.

3. The electronic blood pressure meter according to claim 2, wherein the parameter is at least one of a number of pressure changes, a time interval of a pressure change, a degree of each pressure change, and a maximum pressure value or minimum pressure value in each pressure change.

4. The electronic blood pressure meter according to claim 2,
wherein the processing unit determines that the change in the internal pressure in the fluid bladder detected by the sensor is the predefined change and starts the adjustment performed by the adjustment mechanism in a case where a number of changes in the internal pressure in the fluid bladder detected by the sensor is a number stored in advance,
wherein a time interval of the number of changes is greater than or equal to a specified time stored in advance,
wherein a degree of each change in the internal pressure in the fluid bladder detected by the sensor is greater than or equal to a degree of change stored in advance, and
wherein a maximum pressure value in each change in the internal pressure of the fluid bladder detected by the sensor is greater than or equal to a pressure value stored in advance.

5. A control method for measuring a blood pressure using an electronic blood pressure meter, the electronic blood pressure meter including a fluid bladder for attachment to a measurement area of a measurement subject and a fluid holding portion, into and from which fluid can flow from and to the fluid bladder, that has a lower volume than the fluid bladder, and the fluid being capable of flowing from the fluid holding portion into the fluid bladder in a case where a predetermined pressure is applied to the fluid holding portion, and the control method comprising:
a step of detecting a change in an internal pressure in the fluid bladder prior to a start of a blood pressure measurement operation;
a step of comparing the change in the internal pressure in the fluid bladder with a predefined change;
a step of starting control of the internal pressure in the fluid bladder in the case where the change in the internal pressure in the fluid bladder is the predefined change;
a step of calculating a blood pressure value of the measurement subject based on the change in the internal pressure in the fluid bladder detected during the control of the internal pressure in the fluid bladder; and
a step of outputting the calculated blood pressure value.

6. The electronic blood pressure meter according to claim 3,
wherein the processing unit determines that the change in the internal pressure in the fluid bladder detected by the sensor is the predefined change and starts the adjustment performed by the adjustment mechanism in a case where a number of changes in the internal pressure in the fluid bladder detected by the sensor is a number stored in advance,
wherein a time interval of the number of changes is greater than or equal to a specified time stored in advance,
wherein a degree of each change in the internal pressure in the fluid bladder detected by the sensor is greater than or equal to a degree of change stored in advance, and
wherein a maximum pressure value in each change in the internal pressure of the fluid bladder detected by the sensor is greater than or equal to a pressure value stored in advance.
